(19) United States
(12) Patent Application Publication
(10) Pub. No.: US 2009/0041632 A1
(43) Pub. Date: Feb. 12, 2009

(54) Air Purifier System and Method

(75) Inventors: Edwin David Day, Calgary (CA); John Douglas Pink, Calgary (CA); Robert Brian Knight, Calgary (CA)

Correspondence Address: NOVAPURE SYSTEMS, INC., BAY 6, 3530 - 11A STREET NE, CALGARY, AB, T2E-6M7, CA

(73) Assignee: Novapure Systems Inc., Calgary (CA)

(21) Appl. No.: 11/835,899

(22) Filed: Aug. 8, 2007

(51) Int. Cl. A61L 9/20

(52) U.S. Cl. 422/121

(57) ABSTRACT

The invention provides an air purification system and method for combining filtration, ultraviolet irradiation, and photocatalysis, in an annular geometry that maximizes the photocatalytic efficiency of the ultraviolet light in the deactivation of microbes and the photocatalysis of volatile compounds.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The present invention relates to a system and method for efficiently purifying indoor air by combining forced (variable speed) air movement (fan and motor), filtration, ultraviolet germicidal irradiation, and photocatalysis in an air purifier unit.

Some of the earliest published references to titania (titanium dioxide or TiO₂) photocatalysts are by Formenti, M., et al., “Heterogeneous Photocatalysis for Oxidation of Paraffins”, Chemical Technology1, 680-686, 1971 and U.S. Pat. No. 3,781,194 issued Dec. 25, 1973. Since the 1972 discovery, by Fujishima and Honda, of the photocatalytic splitting of water on titanium dioxide electrodes, the science and technology related to heterogeneous photocatalysis in both water and air has been extensively studied and is the subject of numerous patents and scientific publications. Both the physics and chemistry of heterogeneous photocatalysis remain areas of active investigation. Much of the early work of relevance to this patent is summarized, by Kittrell in U.S. Pat. Nos. 6,179,972 and 6,221,259, as well as, Peill, et al., in U.S. Pat. Nos. 5,875,384 and 6,051,194. Despite investigation of many alternatives, the anatase crystal morphology of titanium dioxide remains the photocatalytically active semi-conductor of economic choice, although many claims of additive enhancements have been and continue to be made. Considerable historical effort has been expended to maximize photocatalytic activity of anatase TiO₂ by minimizing particle size (to maximize effective surface area) while maintaining strong adhesion to substrate surfaces. It is well known and documented that photocatalytic activity is directly related to (a) the intensity and wavelength of irradiation at the illuminated photocatalyst surface area, (b) the magnitude of the illuminated photocatalyst surface area, (c) the rate of flow of contaminants across the illuminated photocatalyst surface area (irradiated surface contact), and (d) in air, the “absolute” humidity of the ambient air. The “quantum or photocatalytic efficiency” relates to the fraction of light-source photons that are effective in causing the photocatalyzed reactions. Considerable effort is currently being expended, in the field of photocatalysis, to enhance the photocatalytic efficiency of anatase titanium dioxide with various catalytic additives (as described in many of the patents cited, e.g., U.S. Pat. Nos. 6,409,928, 6,179,972, and 6,221,259) and to extend the wavelength of photocatalyst-activating irradiation into the visible wavelength range, as described in U.S. Pat. Nos. 7,153,808 and 7,175,911. The objective of the present invention is to provide a large illuminated photocatalyst surface area while ensuring intimate air-surface contact, at all air flow rates, so as to maximize the photocatalytic efficiency of the light source. Where the photocatalyst substrate fibers are high-purity quartz, wool, mat, or felt, the fibers act as elementary waveguides having a UV-conductive core with a reactive outer coated surface. Refraction and reflection (both internal and external) of randomly oriented fibers ensure efficient distribution of light photons throughout the fiber mass, until absorption at the photocatalytic coating occurs.

Early work with photocatalyst powder coatings encountered particle size minimization and bond-to-substrate issues. Various high temperature coating application techniques have been technically successful but are economically and practically prohibitive for coating fiber wool, mat, or felt. Organic binders for powders, such as methylmethacrylate and various organic resins, have been found to emit unacceptable odors under UV-C irradiation. Titanium oxide films formed by inorganic peroxotitanium hydrate sol gels together with a peroxotitanic acid binder have been found to have good substrate-bonding and photocatalytic properties, while remaining odor-free under UV-C irradiation.

Photocatalyzed reactions of volatile compounds (VCs) are known to be strongly endothermic, such that the photocatalyst-activating energy output of commonly available lamps limits the concentrations of treatable airborne VCs to parts-per-million (ppm) or less. The concentrations of most noxious/offensive odors and airborne pathogens are within this treatable range.

U.S. Pat. No. 5,330,722 discloses, in one embodiment, a longitudinal arrangement of a disposable, hollow, cylindrical air filter rotated about the longitudinal axis between two closely spaced UV lamps (one irradiating the inside wall and one irradiating the outside wall. Electrostatic but no photocatalytic enhancement is claimed. Air flow, as in the present invention, is from the outside to the interior (core cavity) of the filter cylinder. The germicidal filter medium is claimed to include fibrous materials (including fiberglass) and pleated paper suitable for use in filtering air. The ultraviolet radiation sources claimed include ozone producing lamps. There is no discussion of UV-induced deterioration of filter materials.

U.S. Pat. No. 5,616,172 discloses a longitudinal but rectangular arrangement of UV lamps within the enclosed housing of an air treatment system. There is neither photocatalytic activity nor disposable cartridge claimed.

U.S. Pat. Nos. 5,779,912 and 6,409,928 disclose a photocatalytic method and apparatus for mineralizing organic contaminants in water or air. The photocatalytic performance is claimed to be enhanced by addition of an oxidant stream to the contaminated fluid stream as well as addition of metal catalysts to the photocatalyst coatings. With some similarities to the present invention in concept, the differences are: (a) the focus of these patents upon contaminated water, (b) the photocatalyst coating is applied only to the directly irradiated surface, and (c) a supplementary oxidant (oxygen or ozone) stream is directed to the reaction zone. Many reasons are stated for rejecting an annular photoreactor concept, such as the present invention.

U.S. Pat. No. 5,833,740 discloses a longitudinal, coaxial combination of UV-V and UV-C light sources within the enclosed housing of a chemical and biological air purifier. There is neither photocatalytic activity nor a disposable cartridge claimed.

U.S. Pat. Nos. 5,875,384 and 6,051,194 disclose the construction and performance characteristics of oriented TiO₂-coated fiber optic cable reactors (FOCRs) and their use in the degradation of organic and inorganic pollutants in both air and water. The fibers described were 400 to 1,000 microns in diameter and were maintained straight and parallel within the reactor. Photocatalyst particles were baked onto the fiber surfaces after dipping into a TiO₂ slurry. Due to weak photocatalyst-fiber bonding, there was a concern for de-lamination of the coating on fiber-to-fiber contact. Therefore, spacers were utilized to prevent fiber-to-fiber contact. The UV light source was unspecified.

U.S. Pat. No. 5,997,619 discloses an annular filter arrangement preceding UV germicidal irradiation. No photocatalysis is claimed.

U.S. Pat. No. 6,053,968 discloses an annular portable room air purifier in which the germicidal UV lamps are arranged at the outer periphery of the core cavity, not on the longitudinal axis. HEPA filtration but not photocatalysis is claimed.

U.S. Pat. Nos. 6,179,972 and 6,221,259 disclose a two-stage process, catalyst, and apparatus for photocatalytic conversion of contaminants in oxygen-containing air and water streams combining a UV-activated photocatalysis stage and a subsequent high temperature catalysis stage. The photocatalyst is described as including various combinations of titanium and zirconium, both supported by silica “gel”.

U.S. Pat. Nos. 6,235,401, 6,344,277, 6,344,278, and 6,379,811 disclose the method of preparation of a yellow transparent jelly (viscous) amorphous type titanium peroxide sol which serves as an excellent binder for titanium dioxide powders and sol gels. These patents further teach that when the titanium peroxide sol is heated at 100 degrees C. or more for several hours, the anatase type of titanium oxide sol is obtained. When a substrate is coated with the amorphous type titanium peroxide sol and then dried and heated at 250 to 940 degrees C., an anatase type of titanium dioxide is obtained. This material is similar to the preferred photocatalyst material of the present invention (without the necessity of heating).

U.S. Pat. No. 6,309,611 discloses a multi-stage photocatalytic reactor for both gaseous and aqueous streams, each stage of which is somewhat similar in concept to the present invention with the following differences:
1. The air flow through the photocatalytic medium is reversed (out from the illuminated side as opposed to into the illuminated side).
2. There is no described pre-filtration.
3. The preferred photocatalytic medium substrate is a cellulosic-fiber fabric material (cotton or any biopolymeric) to which the titania particles are claimed to be hydrogen bonded in a flexible stocking or a rigid metallic or ceramic screen.
   This patent provides an excellent background discussion of prior relevant art, patents, UV light sources, mass transfer considerations, photocatalytic media, and provides an internal classification (by type) of catalytic media.

U.S. Pat. No. 6,358,374 discloses an elaborately schematic integrated photocatalytic gas purifier and an adsorbent bed (gaseous contaminant accumulator). The contaminants are first adsorbed (accumulated/concentrated) in the bed until saturated. The adsorbent bed is then regenerated by heating to release the captured contaminants which are released into a fixed volume of gas that is re-circulated through the heated bed and through a photocatalytic gas purifier which oxidizes the released contaminants. Unfortunately, the patent discloses no details of the photocatalytic gas purifier, light source, or photocatalyst.

WO/2002/004036 discloses a method and apparatus for air purification that consists of a cylindrical arrangement of an inner number of UV light sources (illumination section), preceded by pre-filters (pre-sterilization section). An antimicrobial agent may be coated on surfaces in one or both the filter and illumination sections. Design-induced turbulence is claimed to increase the dwell time of contaminants in the illumination section. However, dwell time is determined by flow rates, not turbulence. Turbulence does increase the possibility of surface contact. No photocatalysis is claimed.

U.S. Pat. No. 6,358,374 discloses a method for preparing a thin film of oriented anatase crystals on a substrate by spraying a vaporized titanium alkoxide (in an inert carrier gas) onto the heated substrate surface, at atmospheric pressure. This method and process is not used in the present invention.

U.S. Pat. Nos. 6,228,502 and 6,465,042 disclose a chemical vapor deposition technique for producing an oriented crystalline film of anatase titanium dioxide on a substrate surface, followed by annealing in an oxygen atmosphere, and then coating with silver, or copper, or oxide thereof.

WO/2002/083307 discloses an annular photocatalytic air purifier comprising: (a) a tubular housing having an inner and an outer wall, a central axis, a first end having a centrally located air intake nozzle, a second end having at least one air exhaust port; (b) an air exhaust plenum between the inner housing wall and a radial porosity medium, the porosity medium extending radially and axially about the axis; and (c) a housing central portion defined by an interior perimeter of the radial porosity medium, the central housing enclosing an ultraviolet lamp and a packing medium, the packing medium extending radially and axially about the lamp and comprising a plurality of spiral wound filaments (semi-transparent to UV light, E-glass, a form of fiberglass, and of 15-25 microns diameter) coated with a photocatalytic film. The substrate/support fibers are claimed to be any of glass, metal, plastic, nylon, or other material that can be assembled into small fibers. Such fibers are claimed to be organized into a twisted strand “bottle brush” configuration, bound by at least one stainless steel wire. Although geometrically similar to the present invention, the major differences are:
1. No pre-filtration is claimed, in contrast with the present invention.
2. The fiber orientation is regular (“bottle brush”), not random (wool, felt, mat), as in the present invention.
3. The fibers are, at best, semi-transparent to long wavelength UV light and opaque to short wavelength UV (germicidal) and not fully transparent, as in the present invention.
4. The direction of air flow is inside-out, not outside-in.

WO/2002/102497 discloses a photocatalytic air purifier apparatus that consists of an annular arrangement of an inner photocatalyst-coated transparent sleeve (“member”) enclosing the axial UV light source with both enclosed by either a photocatalytic cylindrical surface or “a plurality of tubes”. Air flow is directed through the annulus between the transparent sleeve and the outer photocatalytic cylinder. Photocatalytic activity is limited by the smaller UV-illuminated photocatalyst-coated surface area and lesser intimate contact between airborne molecules and the smaller photocatalyst surface than in the present invention.

U.S. Pat. No. 6,531,100 discloses various metal wire mesh, cloth, and non-woven substrates, surface enhanced with particles of many materials bound to the substrate surface, on coating with photocatalyst, to maximize the area presented to a UV light source. Baking or sintering, after wet application of photocatalyst sol, is said to provide a secure photocatalyst bond to the substrate material. The patent also describes various arrangements of planar photocatalyst-supporting bodies and UV light sources in photocatalytic apparati.

U.S. Pat. No. 6,589,489 discloses an air purifier of similar geometry to the present invention but incorporating a “dielectric body” in place of the photocatalytic cartridge of claim 1 and utilizes both UV1 (UV-V) and UV2 (UV-C) light. In this invention, the dielectric body may be made of, for example, quartz, or alumina fibers or silica granules or sponge so that it is porous to air and transmissive to UV light. The dielectric body of this invention may contain photocatalytic material and electron transfer is claimed to be enhanced by and electric field imposed by concentric anode and cathode metal mesh cylinders incorporated within the dielectric body. Air flow is from the inside→out (of the dielectric body) in contrast with the flow from the outside→in (of the photocatalytic cartridge) in the present invention.

U.S. Pat. No. 6,602,918 discloses processes for producing titanium oxide coating agents for the purpose of forming a titanium oxide film on a substrate with improved adhesion and increased density. The product of this invention is very similar to that used in the present invention.

U.S. Pat. No. 6,730,265 discloses an air UV disinfection device and method involving a UV light source connected by fiber optics to a gas purification zone (reaction chamber of gas purifier) that may involve reflective or photocatalyst-coated interior surfaces. This patent also claims “particle arresters” (fiber filters) composed of fibers selected fro the group consisting of glass fibers, acrylic fibers, quartz fibers, paper fibers, cellulose fibers, cotton fibers, plastic fibers, and cominations thereof. Gas flow and control of that flow through the gas purification zone is not detailed.

U.S. Pat. No. 6,602,918 discloses an small (suitable for plugging directly into an electrical outlet) apparatus and method for purifying air consisting of a chimney, UV light source, a doped (platinum metal, etc.) photocatalyst (primarily titanium dioxide) coated on internal walls or fibrous mass (unspecified). Air flow through the unit is maintained by convection created by internal heating. Convection alone is a slow process that generates a small number of air exchanges per hour in even modest-sized indoor environments.

U.S. Pat. No. 6,764,655 discloses a light-leakage type photocatalytic filter comprised of longitudinally bundled photocatalyst fibers with inter-fiber gaps to permit fluid communication pathways. Light introduced at the ends of the fibers travels along the photocatalyst fibers while partially leaking therefrom and thus causing photocatalytic reactions. According to the patent, fluid flow may be parallel, perpendicular, or inclined with respect to the longitudinal direction of the fiber bundle. A bundle consists of about 10,000 fibers of 125 nm in diameter and 200 mm in length. Neither the photocatalyst nor the composition of the fibers is disclosed.

U.S. Pat. No. 6,773,683 discloses a schematic concept for photocatalytically oxidizing sulfur dioxide to sulfur trioxide using a UV laser or lamp light source connected, via fiber bundles, to a reactor but without disclosure of photocatalyst or details of reactor design.

U.S. Pat. No. 6,783,578 discloses an air purification apparatus that includes a HEPA or ULPA cartridge filter and UV lamp configuration, geometrically similar to the present invention but without a photocatalytic medium and with the air flow reversed (from the inside of the cartridge to the outside).

U.S. Pat. No. 6,835,679 discloses a method and apparatus for light curing of composite materials incorporating one or more “lossy” optical fibers. This patent teaches that optical fibers may be made lossy by bending the fiber, weaving the fiber into a mat (to create periodic micro bends), or by removing the fiber cladding. Such light losses (leakage) distribute the light throughout the composite material.

U.S. Pat. No. 6,884,399 discloses a modular photocatalytic air purifier intended for installation in heating, ventilating, and air conditioning systems (fan coil units). UV lamps are positioned between photocatalyst-coated filter elements such that the filter elements serve as the photocatalytic surfaces. The filter geometry is stated to include honey-combs, fins, mesh, a filter-type structure, a fibrous type, or a filamentous structure.

U.S. Pat. No. 6,884,752 discloses an aqueous composition of titanium dioxide that includes varying amounts of acrylic aliphatic urethane polymer to improve wetability of the substrate, as well as, thinness and clarity of the produced film.

WO/2005/039659 discloses a germicidal air treatment method and device for air purification that consists of a longitudinal arrangement of UV light sources, inside an impermeable-walled enclosure, preceded by serial pre-filters (pre-filter, HEPA filter, and possibly a carbon filter), and equipped with a fan and motor. An ionizer is located downstream of the fan. UV irradiation is claimed for at least one side of one filter. The UV irradiation field within the UV treatment chamber is maximized by highly reflective walls (rough-surfaced aluminum, in the preferred embodiment). Components are arranged for turbulent air flow. This invention relies on filtration in place of photocatalysis, unlike the present invention.

U.S. Pat. No. 6,902,653 discloses fluid-permeable reticulated or packed bed photocatalytic reactors in which both the substrate and semiconductor photocatalyst are semi-transparent to the activating light. Fluid flow is either parallel or perpendicular to the lamp axis. The light sources are embedded within the packed bed.

U.S. Pat. No. 6,939,397 discloses a UV-based air-purifying apparatus of similar geometry to the present invention but involving an impermeable-walled, non-filtering, reflective-surfaced cartridge without any photocatalysis.

WO/2006/018949 discloses an air purifier and method for air purification that consists of a tubular (circular or polygonal section) main body having a plurality of air movement holes on the wall surrounding the inner space and an internal UV light source illuminating the inner wall surface. The formed, porous main body may be made of any photocatalyst-supporting fiber material, including cellulose and acrylic fibers. By forming projections and recesses on the inner surface, the irradiated surface area is increased. Air movement (in and out of the air movement holes), in the preferred embodiment, is provided (slowly) by convection resulting from lamp heat and diffusion. However, some forced air movement is suggested to be provided near the air movement holes by a discharge or intake fan. No filtration is claimed.

U.S. Pat. No. 7,063,820 discloses a method for disinfecting fluids on photocatalytic surfaces excited by UV-A irradiation within heating, ventilating, and air conditioning systems or in air ducts. An electrical bias applied to the photocatalytic elements is claimed to enhance the photocatalytic activity at low ambient humidity.

U.S. Pat. No. 7,083,659 discloses a combined light source and non-photocatalytic air purifier unit that a cylindrical filter surrounding the fan. Therefore, the air flow through the filter is the reverse of the present invention and no photocatalysis is involved.

U.S. Pat. No. 7,144,840 discloses a method of preparation of small titanium dioxide crystals in a sol using polyethylene glycol as a dispersing agent before coating substrates and calcining the coated substrate.

U.S. Pat. No. 7,175,814 discloses an annular air disinfecting system and cartridge device containing ultraviolet light. While of similar annular geometry to the present invention, no photocatalysis is contemplated in this patent (only reflective surfaces). In this patent (unlike the present invention), the light source and other electronics are incorporated into the cartridge unit.

US2007084350 discloses a self-contained photocatalytic air purification apparatus comprised of a outer housing with air inlet and outlet ports enclosing sets of UV light sources illuminating both sides of one or more internal filters. Internal surfaces may be polished metal or coated with photocatalyst. Air flow is directed around a series of air baffles “to cause turbulence and to maximize exposure time.” Exposure times are determined by air flow rates, not turbulence. Nevertheless, turbulence does improve the possibility of surface contact. In this invention, photocatalytic activity is limited by the smaller UV-illuminated photocatalyst-coated surface area and lesser intimate contact between airborne molecules and the smaller photocatalyst surface than in the present invention.

U.S. Pat. App. No. 20060086252 discloses a ceiling-mounted, single-sided, planar arrangement of UV lamps, photocatalytic filter, and active carbon coated filter in an air purifier. No detail is provided regarding the lamps or photocatalyst.

U.S. Pat. App. No. 20060086252 describes a photocatalytic air purifier comprised of a conventional lamp and shade arrangement in which the inner surface of the lamp shade is coated on the inner illuminated surface with a photocatalyst (broadly specified) such that heat generated by the light source (broadly specified) causes convection of air past the lamp shade surface. Convection alone is a slow process that generates a small number of air exchanges per hour in even modest sized indoor environments.

### BRIEF SUMMARY OF THE INVENTION

In an annular arrangement of components, this invention comprises an outer housing that encloses a fan and motor, and a cylindrical photocatalytic cartridge concentrically/coaxially enclosing a light source illuminating the entire inside area and volume (core cavity) of the cartridge. In a preferred embodiment, the order of photocatalyst cartridge media/elements from the outside wall to the inside are: outer protective mesh (metal or plastic), pre-filter medium, photocatalyst medium, and innermost structurally rigid metal mesh (preferably reflective aluminum). The fan and motor may be sized to provide the desired compromise between air flow rate, the air flow resistance of the photocatalytic cartridge, and air-rush noise.

The unit housing, photocatalytic cartridge and light source are selected to provide the desired ultraviolet germicidal irradiation dosages (residence time inside the cartridge core cavity) at the prescribed air flow rates while maximizing the photocatalytic surface contact between the flowing air and the interiorly illuminated photocatalyst-coated cartridge medium (turbulent flow ensures intimate air-surface contact).

Centrifugal, axial, or other fans may be incorporated in this invention. However, backward-tipped axial fans have excellent pressure profiles and noise ratings, and are incorporated in a preferred embodiment of this invention.

### BRIEF DESCRIPTION OF THE DRAWINGS

The various features of the present invention may be more fully understood with reference to the following description and the accompanying drawings in which:

FIG. 1 is a schematic diagram indicating one embodiment of an assembled unit (including air flow) and the major component parts.

FIG. 2 is a schematic cross-sectional diagram showing the components of one embodiment of a photocatalytic cartridge.

FIG. 3 is a schematic diagram illustrating “longitudinal light-in-pipe” parameters used to calculate the cumulative ultraviolet germicidal irradiation dosage in an air stream flowing through a cylinder/pipe parallel to a linear light source located on the cylinder/pipe axis.

FIG. 4 is a schematic diagram illustrating the coating process of a quartz fiber photocatalyst substrate, according to one embodiment of the present invention.

FIG. 5(a) is a scanning electron micrograph (SEM) of photocatalyst-coated 9 micron diameter (average) quartz fibers at 100× magnification.

FIG. 5(b) is an SEM of the same photocatalyst-coated 9 micron diameter (average) quartz fiber sample at 500× magnification.

FIG. 6(a) is an SEM of a single un-coated quartz fiber at 15,000× magnification.

FIG. 6(b) is an SEM of a single photocatalyst-coated quartz fiber at 15,000× magnification.

FIG. 6(c) is an SEM of a single un-coated quartz fiber at 70,000× magnification (near the limit of clear resolution).

FIG. 6(d) is an SEM of the broken end of a coated quartz fiber, at 15,000× magnification, to show that the coating thickness is a very small fraction of the fiber diameter.

FIG. 7 is an energy-dispersive x-ray spectroscopy (EDS) spectrum (elemental analysis), of the coated fibers.

### DETAILED DESCRIPTION OF THE INVENTION

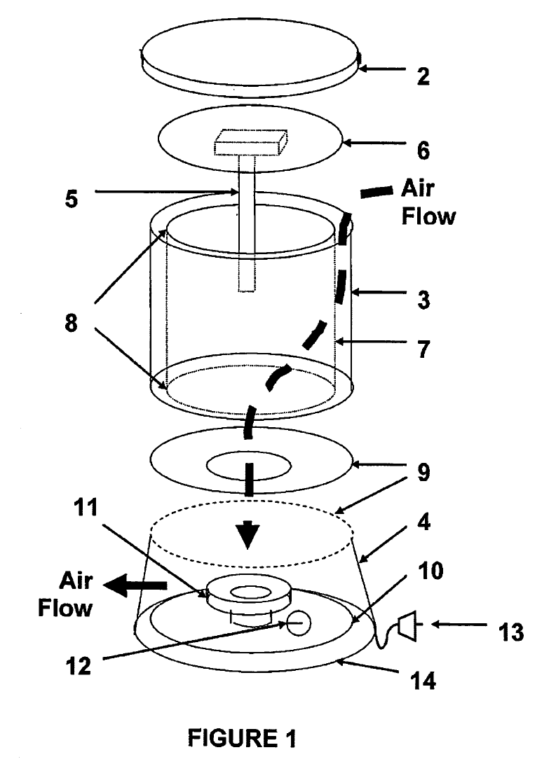

FIG. 1 is an illustrative schematic diagram of one embodiment of an air purifier system and unit according to the present invention. The system 1 generally includes the housing (top 2, sides 3, and base 4 with air exit ports or louvers), a photocatalyst-activating light source 5 and light source mounting plate 6, photocatalytic cartridge 7 with elastomeric gasket rings 8, photocatalytic cartridge end-seal plate 9 (with central port), electrical component mounting plate 10, fan and motor 11, speed control/switch 12, power cord 13, and base plate 14. When assembled, the mounting plate 6 and cartridge end-seal plate 9 form air-tight seals with the elastomeric gasket rings 8, shown as part of the photocatalytic cartridge 7.

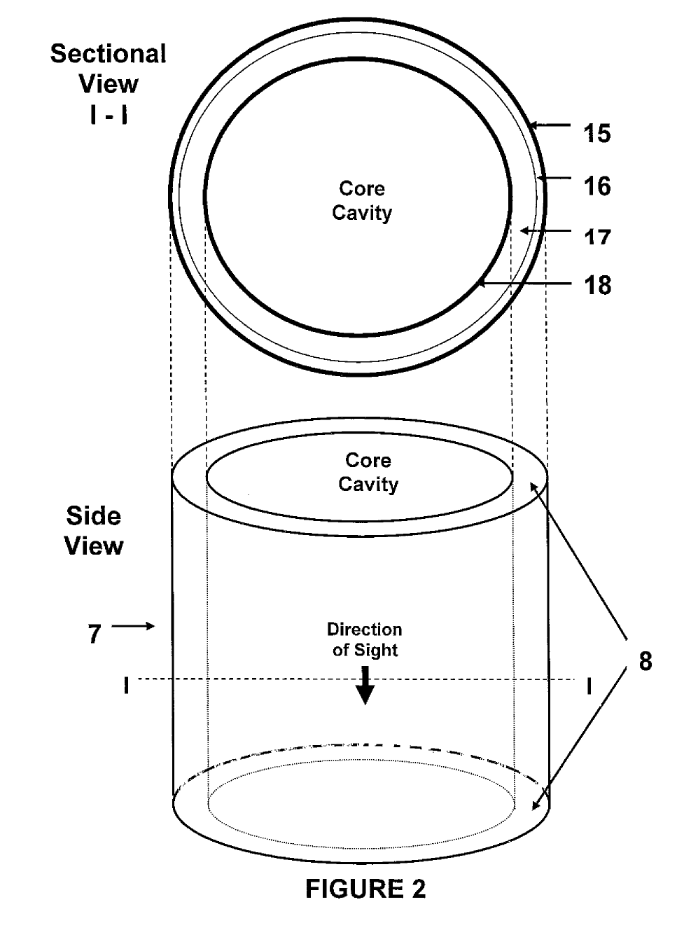

FIG. 2 is a schematic diagram of components making up a preferred embodiment of the photocatalytic cartridge of claim 1. All elements are sealed inside the end enclosures (elastomeric rings 8, in a preferred embodiment) with an odor-free sealant 15. The outer mesh 15 (plastic or metal), in the preferred embodiment, serves only to physically protect the pre-filter medium 16 and the photocatalytic medium 17 and may provide little or no structural strength. Mesh “open areas” should be large enough so as not to restrict air flow. Concentrically inside or outside any protective mesh 15, is the pre-filter medium 16, pleated or un-pleated. A pleated pre-filter may be required (low air flow resistance) for very high air flow rates. However, un-pleated pre-filter media are adequate for most applications. The MERV rating of the pre-filter medium should be no less than 8 to keep clean the next-inside photocatalytic medium 17. In a preferred embodiment, the substrate material of the photocatalytic medium is a high-purity quartz fiber, wool, mat, or felt. However, a highly reflective metal wool, open-cell metal sponge, or open-cell metal foam, (e.g., of stainless steel or aluminum) could also be an effective substrate medium. The dominant photocatalytic substrate medium selection criterion is the survival of the UV light photons until absorbed by the photocatalytic coating. Non-conductive and non-reflective substrates do not permit penetration of the photons beyond the first encountered surface (any “shadowed” material is without photocatalytic effect). The finer are the fibers per gram, in the preferred quartz fiber embodiment, the larger is the substrate surface area available for photocatalysis. Ultra fine (2 micron diameter), fine (4 micron diameter), and coarse (9 micron average diameter) high-purity quartz fiber wool, mat, and felt materials are examples of such substrate media.

The innermost element of the photocatalytic cartridge is a rigid metal mesh 18 that provides the structural resistance to compression that permits formation of an air-tight seal between the plates 6 and 9 and the end ring closures 8 of the photocatalytic cartridge 7. In another embodiment, within the spirit of the present invention, the end ring closures 8 could be made of a non-gasket (non-elastomeric) material, with gaskets permanently fixed to the plates 6 and 9.

The biocidal performance of the present invention is the sum of two independent processes: (a) the ultraviolet germicidal irradiation, and (b) photocatalytic oxidation and reduction at or near the photocatalyst surface (the reaction zone). Photocatalysis alone is both germicidal and breaks down VCs into harmless reaction products, as understood and described in the prior art.

### Ultraviolet Germicidal Irradiation

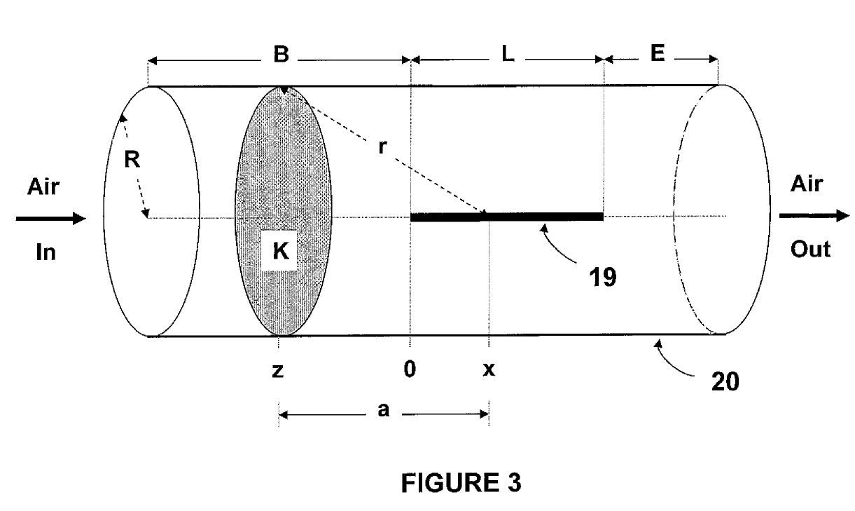

To be germicidal, the wavelength of the radiation must be sufficiently short (energetic) to break chemical bonds or, at least, denature the DNA or proteins of microbes. This is generally accepted to be in the UV-C and UV-V ranges of the electromagnetic spectrum. The “average” ultraviolet germicidal irradiation dosage (energy per unit area irradiated) within the photocatalytic cylinder core cavity may be estimated by first calculating a longitudinal “light-in-pipe” dosage and then dividing that result by two to account for the fact that the average transit (residence) time of a steady-state flow of air, F (cubic feet per minute), entering through the permeable walls is one-half the transit/residence time of an equivalent flow of air entering in one end of the core cavity and exiting out the other. FIG. 3 is a schematic representation of a linear UV light source 19 (of length L) located on the center line (x-axis) of a cylinder 20 (of radius R and length B+L+E, open to air flow only at the ends). Determination of the UV dosages developed by the UV light source in this configuration involves two sets of calculations: (a) a determination of the cumulative UV power density (watt/cm²) incident upon a surface K of area πR², oriented perpendicularly to the UV light source axis and located at any position “z” along the center line of the cylinder, and then (b) a calculation of the cumulative UV energy (watt-second/cm² or joules/cm²) incident upon that surface K (both sides) as it moves through the cylinder from one end to the other, at the same linear velocity as the air flow.

If the inlet end of the light source is considered to be at the origin (zero) of the x-axis, then −B (negative B) is the x-coordinate of the inlet end of the cylinder, L is the x-coordinate of the outlet end of the light source, and L+E defines the x-coordinate of the outlet end of the cylinder 20.

### UV Power Density

If the UV output of the light source is W watts, evenly distributed along its length, L, then each infinitesimal element, dx, of the UV source at a position “x” along its length may be considered as a “point source” of UV light, radiating uniformly in all directions. The infinitesimal UV output, dW, attributable to this element, dx, is:

dW=(W/L)dx

If the surface K is located at position “z”, a distance “a” to the left of “x”, the total sphere area, S, of radius “r” surrounding the point at “x” is

S=4πr², and

the area of the “slice” of that sphere with base K defined by the solid angle subtended by K at x is

A=2πr(r−a),

such that the fraction of the total irradiance from dW at x, falling on K is

$$\begin{matrix}A / S = ( r - a ) / 2 r \\ = 0.5 * ( 1 - a / r ) .\end{matrix}$$

But a=x−z and r²=a²+R², so that

A/S=0.5*{1−(x−z)/[(x−z)²+R²]^{1/2}},

and the average power density, dI_{z}, incident upon K from dW at x is

dI_{z}=(A/S)dW/K watts/cm²

Integrating over all infinitesimal elements dI_{z} of the UV light source (from x=0 to x=L), yields the average power density (watts/cm²) received by surface K at position “z”.

$$\begin{matrix}{I}_{z} = \int d {I}_{z} \\ integrating over the whole bulb length ( x = 0 to L ) . \\ = ( W / ( K * L ) ) \int ( A / S ) d x , \\ = ( W / ( 2 * K * L ) ) * \{ L - {[ {( L - z )}^{2} + {R}^{2} ]}^{1 / 2} - {[ {z}^{2} + {R}^{2} ]}^{1 / 2} \}\end{matrix}$$

### UV Energy Dosage

If the steady-state air flow rate is F (cubic feet per minute or “cfm”), the average linear velocity of the air and K is F/K (feet per minute, where K is measured in square feet). Therefore, the transit time for air to traverse the cylinder/pipe, i.e., K to travel from −B to L+E along the x-axis is (B+L+E)*K/F. Therefore, the cumulative UV dosage (watt-sec./cm² or joule/cm²), CD, delivered by the UV light source and received by area K traversing the cylinder/pipe is the sum of three parts: the two single-sided end contributions, CD_{B} and CD_{E}, and the two-sided (both sides of K) contribution at the bulb, CD_{L}.

$$\begin{matrix}CD = {CD}_{B} + {CD}_{L} + {CD}_{E} \\ = \int {I}_{z} d t ,\end{matrix} \\ integrating between \\ t = - BK / F \\ ( z = - B ) \\ and \\ t = ( L + E ) * K / F \\ ( z = L + E ) . \qquad EQUATION 1$$

But the steady-state linear velocity F/K=dz/dt=dx/dt, so that dt=(K/F)dz, and so

$$\begin{matrix}{CD}_{B} = ( K / F ) \int {I}_{z} d z , integrating between z = - B and z = 0 \\ = ( W / ( 2 * F * L ) ) * \{ B * L + 0.5 * \\ ( B * {[ {B}^{2} + {R}^{2} ]}^{1 / 2} + L * {[ {L}^{2} + {R}^{2} ]}^{1 / 2} - ( L + B ) \\ {[ {( L + B )}^{2} + {R}^{2} ]}^{1 / 2} + {R}^{2} * ln \{ R * ( L + {[ {L}^{2} + {R}^{2} ]}^{1 / 2} ) / \\ ( ( {B [ {B}^{2} + {R}^{2} ]}^{1 / 2} - B ) * ( L + B + {[ {( L + B )}^{2} + {R}^{2} ]}^{1 / 2} ) ) ) \} )\end{matrix}$$

$$\begin{matrix}{CD}_{L} = 2 ( K / F ) \int {I}_{z} d z , \\ integrating between z = 0 and z = L ( both sides of K ) \\ = W * L / F\end{matrix}$$

$$\begin{matrix}{CD}_{E} = ( K / F ) \int {I}_{z} d z , \\ integrating between z = L and z = L + E \\ = ( W / ( 2 * F * L ) ) * \{ E * L + 0.5 * ( E * {[ {E}^{2} + {R}^{2} ]}^{1 / 2} + L * \\ {[ {L}^{2} + {R}^{2} ]}^{1 / 2} - ( L + E ) * {[ {( L + E )}^{2} + {R}^{2} ]}^{1 / 2} + {R}^{2} * \\ ln \{ ( R * ( L + {[ {L}^{2} + {R}^{2} ]}^{1 / 2} ) / ( ( {[ {E}^{2} + {R}^{2} ]}^{1 / 2} - E ) * \\ ( L + E + {[ {( L + E )}^{2} + {R}^{2} ]}^{1 / 2} ) ) ) \} )\end{matrix}$$

These formulae assume no internal reflection of any photons. For a “permeable-walled” photocatalytic (PC) cartridge, the cumulative UV dosage is one-half the calculated CD. In this dynamical system, only the end dosages (CD_{B} and CD_{E}) formulae involve R, and each are of the same form. Within the length of the bulb (z=0 to z=L), the dosage, CD_{L}, involves only W, L, and F, with no explicit dependence upon K (integrals involving R cancel). While this result is somewhat counter-intuitive, it can be understood by the linear velocity of K as F/K, such that, for example, when K is doubled, the linear velocity of K is halved so the dosage remains the same. When B and E are zero, CD_{B} and CD_{E} are also zero, respectively.

TABLE 1
Illustrative Cumulative UVGI Dosage (CD) Formula Results*

| UV-C Bulb Rating (Watts): | 18 W | | 36 W | | 36 W | |
|---|---|---|---|---|---|---|
| Number of Bulbs: | 2 | | 1 | | 2 | |
| UV-C Output: % | 30.8% | | 30.8% | | 30.8% | |
| UV-C Watts, W | 11.1 | | 11.1 | | 22.2 | |
| Envelope Length, L (Inches): | 7.5 | | 15.0 | | 15.0 | |
| Cartridge/Pipe Parameters: | | | | | | |
| Length, B + L + E (Inches): | 10.0 | | 10.0 | | 10.0 | |
| Distance before Bulb, B (Inches): | 1.5 | | 1.5 | | 1.5 | |
| Distance after Bulb, E (Inches): | 1.5 | | 1.5 | | 1.5 | |
| Internal Radius, R (Inches): | 4.0 | | 4.0 | | 4.0 | |
| Air Flow, F (cfm):* | 50 | 100 | 50 | 100 | 50 | 100 |
| UVGI Dosage, CD (μwatt-sec/cm²): | | | | | | |
| CD_{B} + CD_{E} | 499 | 250 | 292 | 146 | 584 | 292 |
| CD_{L} | 8,951 | 4,475 | 17,903 | 8,951 | 35,805 | 17,903 |
| Impermeable Pipe (CD_{B} + CD_{E} + CD_{L}): | 9,450 | 4,725 | 18,194 | 9,097 | 36,389 | 18,194 |
| PC Cartridge (permeable walls): | 4,725 | 2,362 | 9,097 | 4,548 | 18,194 | 9,097 |

*Note: for a room volume of 1,000 cubic feet, an air flow of 50 cfm provides 3 air exchanges per hour and 100 cfm provides 6 air exchanges per hour.

The results in TABLE 1 are self-consistent to the extent that doubling the air flow rate halves the UV dosage and halving the UV output (one 36 W bulb in place of two) also halves the UV dosage. Furthermore, a longer UV bulb extends the residency time in the radiation field of the UV light source and, hence, the greater UV dosage calculated for one long 36 W bulb versus two short 18 W bulbs. These results also imply consistent units conversions (imperial units to metric units and vice versa). The dosage units are W-sec/cm² (or J/cm²), which must be multiplied by 1,000,000 to convert to the usual units μW-sec/cm² or μJ/cm² as commonly used in the literature. Ninety percent (90% or “one log”) of many airborne species of molds, bacteria, and viruses are killed or “deactivated” at dosages well under 10,000 μW-sec/cm² (or 100 J/m², see References 1-4).

### Photocatalytic Substrate Fiber Area and Coating Density

High purity quartz has a density of 2.20 g/cm³. Therefore, a given weight (grams), W, of quartz fiber material has a volume, V, where

$$\begin{matrix}V = L \times π {R}^{2} , \\ where L is the total fiber length ( cm ) and R is the \\ average fiber cross \text{-} sectional radius ( cm ) \\ = W / 2.20 {cm}^{3} , \\ and \\ L = W / ( 2.20 * π {R}^{2} ) cm\end{matrix} \\$$

The photocatalyst-coatable fiber surface area, A, is then given by the fiber circumference times the length, L:

$$\begin{matrix}A = 2 π R \times L {cm}^{2} \\ = 4 W / ( 2.20 * D ) {cm}^{2} ,\end{matrix} \\ where \\ D = 2 R is the fiber diameter in cm . \qquad EQUATION 2$$

For a coarse quartz wool (9 micron=9×10⁻⁴ cm diameter fibers), each gram of such wool presents a total coatable fiber surface area of 4/(2.20*9×10⁻⁴) or 2.02×10³ cm².

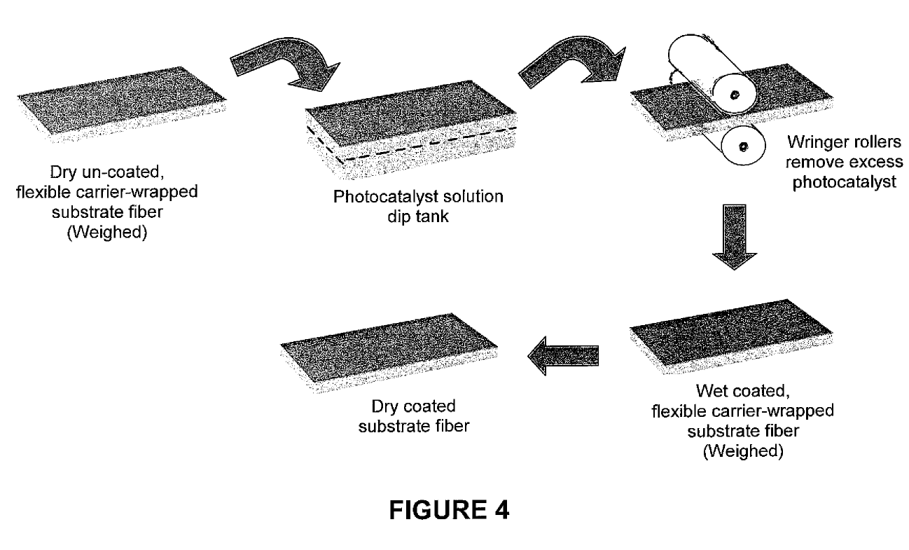

FIG. 4 provides a schematic representation of a dip-coating process for coating a known weight, S_{D}, of dry photocatalytic substrate fiber material (wool, mat, or felt) with a determinable surface coating of photocatalyst starting with a sol gel solution of known concentration, C (e.g., g/ml of anatase TiO₂). Given the density, p, of the sol gel solution and the weight of the sol-gel wet substrate fiber mass, S_{W}, the weight of retained wet photocatalyst coating, PC, may be calculated as:

PC=(S_{W}−S_{D})*C/P grams

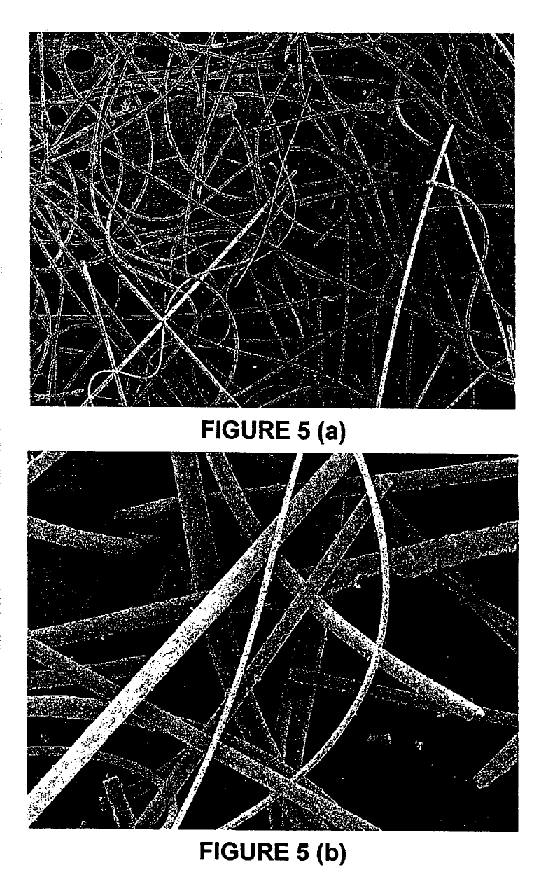

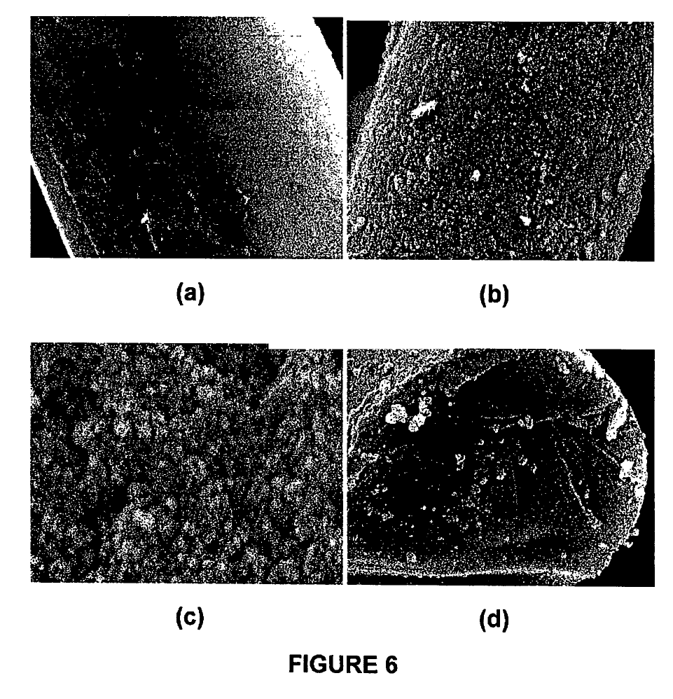

and the average weight distribution per unit area of fiber surface as PC/A. FIG. 5(a) and (b) are scanning electron micrographs of a “nominal” (average) 9 micron diameter photocatalyst-coated quartz wool at 100× and 500× magnifications, respectively. The diversity of fiber diameters and the bends and the breaks in the fibers are evident in these images. Increasing the magnification to 15,000× of one of the un-coated and coated fibers in FIG. 6(a) and FIG. 6(b), respectively, shows the excellent coverage but very “rough” surface of the dried photocatalytic coating (FIG. 6(b)). A further magnification to 70,000× in FIG. 6(c) shows visible “agglomeration” of particles in the 10 to 100 nm range.

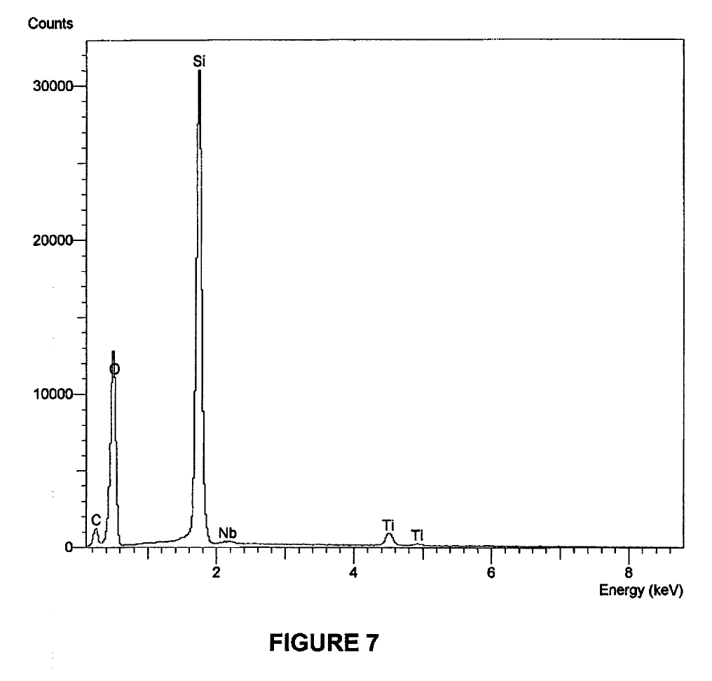

Elemental analysis, using energy-dispersive x-ray spectroscopy (EDS), of the coated fibers is shown in FIG. 7. This analysis shows the expected silicon dioxide and titanium dioxide with “trace” amounts of unexpected carbon and niobium probably due to the sample retaining tape and possibly also due to the sample holder for the analysis.

### Photocatalyst Coating Thickness and Area

A photocatalytic cartridge containing 40 g of nominal 9 micron quartz fiber adsorbed 109.1 ml of 2.20% titanium dioxide sol gel also containing peroxotitanic acid binder with a combined solution density of 1.032 g/ml (0.0227 g/ml anatase sol gel and 0.0103 g/ml peroxotitanic acid binder). The retained sol gel weight implies 2.477 g of TiO₂ (formula weight of 79.87 amu) or 3.101×10⁻² mols. By EQUATION 2, this weight of quartz wool has an estimated surface area of 4*40/(2.20*9)×10⁶ cm² or 8.081×10⁶ cm². Therefore, the formula weight units (mols) per cm² are 3.101×10⁻²/8.081×10⁶=3.837×10⁻⁹ mols/cm². The unit cell dimensions of nanocrystalline anatase (see Weirich, Reference 5) are 3.872×3.872×9.616 cubic angstroms=0.14417 cubic nm or 0.03604 nm³ per TiO₂ unit (four TiO₂ units per anatase unit cell). Therefore, a densely packed “spherical” 10 nm diameter particle would contain approximately 14,528 TiO₂ formula units. Furthermore, given the Avogadro Number of formula units per mol (i.e., 6.022045×10²³), the number of mols/cm² implies 3.837×10⁻⁹×6.022045×10²³/14,528=1.590×10¹⁰ of 10 nm particles/cm². Assuming hexagonal closest packing of spheres, a single layer of 10 nm particles would have an areal packing density of approximately 12×0.5×5×10 nm²=300 nm² per 3 particles or 100 nm² per each 10 nm diameter particle. Each square cm of substrate fiber surface would then accommodate 1/(100×10⁻¹⁴cm² per particle) particles in a single layer or 1×10¹² particles per cm² of substrate surface. This is more than the above calculated 1.590×10¹⁰ particles/cm² applied. This result implies a surface coating with gaps between 10 nm particles or an average “mono-layer” particle size of less than 10 nm diameter, i.e., about 1 nm diameter with about 7 formula units per particle. The greater apparent coverage than calculated for surface density of “compact” 10 nm particles suggests that the agglomerations of FIG. 6(c) are not compact, but loosely packed (possibly porous) and of low volumetric density.

A mono-layer of three-dimensional close-packed spheres (of uniform diameter) on a two-dimensional planar surface have a total sphere surface area to plane surface area ratio of 2π/√3=2.094, independent of sphere diameter. Therefore, an estimate of photocatalyst area on a uniformly covered (no gaps) substrate surface is 2.094 times the substrate surface area. In the example of 9 micron diameter coarse quartz wool (see above), this implies a photocatalyst surface area of approximately 2.094×2.02×10³ cm² per gram of photocatalyst, further enhanced by the distribution of photocatalyst particle sizes and surface roughness. While not all of this photocatalyst surface is accessible to UV photons, errors of over-estimation and under-estimation are expected to approximately cancel each other.

FIG. 6(d) shows a SEM of a cross-section (broken end) of a coated quartz fiber at 15,000× magnification. One millimeter, on the scale of this figure, translates to approximately 100 nm. Therefore, the thickness of the “loosely packed” coating (barely visible at the right-hand edge of the fiber) is estimated to be less than 100 nm. However, silica dust particles on the fibers, prior to coating, further add to (enhance) the coated fiber surface “roughness” and the photocatalytically active surface area.

### Fiber Geometry and Orientation

As discussed by Peill, et al., in U.S. Pat. Nos. 5,875,384 and 6,051,194, as fiber thickness is increased, photons undergo fewer reflections at the quartz-TiO₂ interface for a fixed incident angle and a given length, such that with a thicker diameter fiber, the probability that a photon will be refracted through the quartz-TiO₂ interface is reduced. The small diameter fibers and tangled (many bends) nature of the quartz wool of this invention (as shown in FIG. 5(a) and FIG. 5(b)) enssures a higher probability that a photon will be refracted through the quartz-TiO₂ interface and absorbed by the coating. While Peill, et al., in U.S. Pat. Nos. 5,875,384 and 6,051,194 concluded that chemical efficiency would be enhanced by employing larger diameter fibers in their fiber cable reactor, the opposite is true of this invention.

### Light Intensity and Wavelength Dependence

To be germicidal (without photocatalysis), the UV irradiation should be in the more energetic UV-C to UV-V ranges. As noted in prior art, photocatalytic activity also occurs with less energetic UV-A and UV-B irradiation. Irradiation saturation effects were noted by Peill, et al., in U.S. Pat. Nos. 5,875,384 and 6,051,194, where a four-fold increase in the quantum efficiency (of photo-oxidation/reduction reactions) was achieved with a two-order of magnitude reduction in the absorbed light intensity. However, optimization was stated to be achieved by increasing the fiber number density, which would divide the photon flux among more fibers and thus present a greater photocatalytic area (as achieved by the present invention).

While the foregoing may emphasize the preferred embodiments of the present invention, for illustrative purposes, other and further embodiments may be devised without limiting or departing from the spirit and scope of the present invention, as determined by the following claims.

1. A photocatalytic air purifier system comprising an outer housing, an ultraviolet or near ultraviolet (UV, 100 to 450 nm wavelength range) light source (photocatalyst-activating), air inlet port(s) and air outlet port(s), a variable speed fan and motor, and a cylindrical, replaceable, photocatalytic cartridge located concentrically about the longitudinal axis of the UV light source and closed to air flow at one end, such that air flow is directed inward through the photocatalytic cartridge walls and out the open end.

2. The source of photocatalyst-activating UV irradiation may be any UV generating lamp or columnar array of light emitting diodes (LEDs), but in the preferred embodiment is germicidal.

3. The photocatalytic cartridge of claim 1 consists of an optional (but preferred) metal or plastic protective mesh and a layer of pre-filter material (pleated or un-pleated but which, in a preferred embodiment, is un-pleated with no less than a MERV 8 rating) which encloses a layer of photocatalytic medium wrapped about an inner structurally rigid metal mesh all sealed by end-ring enclosures at both ends (in a preferred embodiment by elastomeric gasket rings) such that only the cartridge core cavity is open at both ends.

4. The photocatalytic medium, in the cartridge of claim 1, consists of a permeable material coated with a photocatalyst, which, in its preferred embodiment, is a microcrystalline anatase titanium dioxide-based coating bound to the medium substrate surface with a UV-resistant inorganic binder (for example, peroxotitanic acid or derivative) and of sufficient photocatalytic medium thickness to provide for complete (or near) extinction of the UV light at the outside surface next to the pre-filter medium, to prevent deterioration of the pre-filter medium.

5. The photocatalytic medium substrate of claim 4, in its preferred embodiment, is a high-purity, UV-transparent high-purity quartz fiber material (felt, mat, or wool) such that all surfaces of the medium are fiber-optically or reflectively accessed by the UV light to maximize the photocatalytic efficiency of the UV irradiation.

6. A tangled (random orientation), fibrous wool, mat, or felt photocatalytic medium substrate of claim 4, in its preferred embodiment, ensures turbulent air flow on passage through the medium and multiple particle/molecular contacts at or near the photocatalytic surface (the reaction zone).

7. The incorporation of high-purity quartz fiber substrate material of claim 4, with known average fiber diameter and weight, permits a calculated estimate of the effective (UV light accessible) substrate fiber surface area available for photocatalysis.

8. The coating of the photocatalytic substrate medium of claims 4 and 5 is effected by (a) wrapping a weighed quantity of the substrate medium in a flexible carrier mesh that remains strong when wet (e.g. fiberglass mesh fabric), (b) immersing the medium and carrier in a photocatalyst sol solution (with surfactant to ensure good coverage), (c) wringing out the excess sol, from the saturated substrate medium in the carrier mesh, between soft rubber rollers, (d) weighing the sol-wet substrate medium to measure sol retention (coverage), and then (e) setting aside the sol-wet substrate medium to dry or bake before incorporation into the cartridge of claim 1.

9. The construction of the photocatalytic cartridge of claim 1 and coating technique of claim 8 precludes de-lamination of the photocatalyst coating from the substrate media under air purification operating conditions.

10. The coaxial geometry of the invention permits scaling of unit dimensions to accommodate a wide range of UV light sources, UV power ranges, photocatalysts, substrate media, air flow rates, and noise level requirements.

11. The air inlet port area, annular area (between the outside of the cartridge and outer housing wall), the fan and fan-outlet port areas are all matched so as not to restrict air flow and to minimize air-rush noise.

12. The photocatalytic cartridge of claim 1 acts to dampen “air rush” noise.

13. The mounting plate for the UV light source(s) closes one end (with one ring gasket) of the cartridge core cavity of claim 1 to force air flow through the interiorly UV-illuminated walls of the cartridge core cavity and out the open end, which butts against a centrally ported plate, with a second ring gasket.

14. The pre-filter at the outside wall surface of the photocatalytic cartridge of claim 1 keeps the UV irradiated inside surfaces “clean” for the economic life of the cartridge (i.e., until air flow resistance created by pre-filter plugging becomes excessive).

15. The components of the air purification system of claims 1 to 14 may be incorporated in fixed, portable, or mobile installations for indoor air or fugitive noxious gas or vapor emissions treatment applications.

16. A formula (EQUATION 1) for estimating the average ultraviolet germicidal irradiation dosage delivered by the UV light source within the illuminated core cavity of the photocatalytic cartridge of claim 1.

17. Formulae for estimating (a) the available fibrous substrate surface area (EQUATION 2), and (b) the photocatalyst coating coverage density of a fibrous substrate within the photocatalytic cartridge of claim 1 (EQUATION 3).
